(19) United States
(12) Patent Application Publication
(10) Pub. No.: US 2010/0099884 A1
(43) Pub. Date: Apr. 22, 2010

(54) METHOD OF PREPARING S-(-)-AMLODIPINE OR A SALT THEREOF AND AN INTERMEDIATE USED THEREIN

(75) Inventors: Sun Young JANG, Suwon-si (KR); Sungbum Kim, Guri-si (KR); Sangmin Yun, Seongnam-si (KR); Hyo Jeong Bang, Yongin-si (KR); Han Kyong Kim, Yongin-si (KR); Kwee Hyun Suh, Suwon-si (KR)

Correspondence Address: SUGHRUE MION, PLLC, 2100 PENNSYLVANIA AVENUE, N.W., SUITE 800, WASHINGTON, DC, 20037, US

(73) Assignee: HANMI PHARM. CO., LTD, Kyungke-do (KR)

(21) Appl. No.: 12/523,957

(22) PCT Filed: Jan. 29, 2008
(86) PCT No.: PCT/KR2008/000530

§ 371 Date: Jul. 21, 2009

(30) Foreign Application Priority Data
Feb. 14, 2007 (KR) 10-2007-0015363

(51) Int. Cl. C07D 213/803

(52) U.S. Cl. 546/321

(57) ABSTRACT

The present invention provides a novel method for preparing S-(−)-amlodipine having a high optical purity or a salt thereof and an intermediate used therein.

### FIELD OF THE INVENTION

The present invention relates to a method of preparing S-(−)-amlodipine or a salt thereof and an intermediate used therein.

### BACKGROUND OF THE INVENTION

Amlodipine of formula (II) is a long-acting calcium channel blocker approved as a commercially marketable therapeutic agent for cardiovascular diseases such as angina pectoris, hypertension, heart failure.

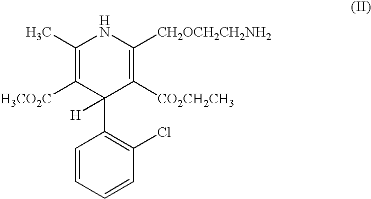

Amlodipine is a racemate composed of equal amounts of S-(−)-amlodipine and R-(+)-amlodipine. The activity of S-(−)-amlodipine of formula (I) is 1000-fold higher or more than R-(+)-amlodipine and 2-fold higher than racemic amlodipine in the calcium-induced contraction of mice aorta (J. E. Arrowsmith et al., J. Med. Chem. 29, (1986), 1696). Thus, it is inferred that the pharmaceutical action of amlodipine as a calcium channel blocker is mostly induced by S-(−)-amlodipine. Also, International Publication Patent No. WO 93/10779 discloses that an optically pure S-(−)-amlodipine is effective in the treatment of hypertension or angina pectoris:

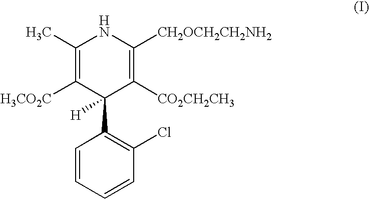

Various methods for preparing optically pure S-(−)-amlodipine have been developed. For example, Europe Publication Patent No. 0,331,315 and [S. Goldmann et al., J. Med. Chem. 35, (1989), 3341] disclose a method for preparing S-(−)-amlodipine by optical resolution using a specific intermediate. However, process steps of this method are complicated.

A method for preparing S-(−)-amlodipine which comprises selectively crystallizing D-(−)-tartate of S-(−)-amlodipine in a solvated form of dimethylsulphoxide is disclosed by International Publication Patent No. WO 95/25722.

Also, International Publication Patent Nos. WO 03/035623 and WO 2006/043148 disclose a method for preparing S-(−)-amlodipine by selectively crystallizing D-(−)-tartate of S-(−)-amlodipine in a solvated form of N,N-dimethylacetamide or N,N-dimethylormamide. Further, International Publication Patent Nos. 01/60799, WO 2005/049571 and Korea Patent No. 0476636 teach a method for preparing S-(−)-amlodipine by subjecting amlodipine to optical resolution to form tartate in a solvent comprising dimethylsulphoxide.

However, the above mentioned methods involving selective crystallization of D-(−)-tartate of S-(−)-amlodipine in a solvated form of dimethylsulphoxide, N,N-dimethylacetamide or N,N-dimethylormamide have problems as described below.

Dimethylsulphoxide, dimethylacetamide or N,N-dimethylormamide, which has a high boiling point of at least 150° C. and is easily miscible with water, cannot be recovered in a pure form after use due to the difficulty of removing contaminant water therefrom by distillation. Thus, after filtrating precipitated tartate of S-(−)-amlodipine, the filtrate containing some uncrystallized tartate of R-(−)-amlodipine must be disposed, e.g. by incineration. Furthermore, the above solvent has a high polarity and tends to adsorb on the product. Such a product must be purified further to meet the purity requirement set by ICH Guideline (ICH Harmonized Tripartite Guideline, Impurities: Guideline for Residual Solvents Q3C(R3), 2006) which strictly limits the residual amounts of such solvents.

Accordingly, the present inventors have attempted to develop a method which uses an organic solvent having a low boiling point and have found a novel method which involves the formation of crystalline complex of S-(−)-amlodipine•D-(−)-tartrate with urea.

### SUMMARY OF THE INVENTION

Accordingly, it is an object of the present invention to provide an improved method of preparing S-(−)-amlodipine having a high optical purity or a salt thereof using complex of S-(−)-amlodipine•D-(−)-tartrate with urea, namely S-(−)-amlodipine•D-(−)-tartrate•urea complex (2:1:1), as a novel intermediate.

In accordance with one aspect of the present invention, there is provided a method for preparing S-(−)-amlodipine of formula (I) or a pharmaceutically acceptable salt thereof, which comprises the steps of:

(i) bringing amlodipine of formula (II) to react with D-(−)-tartric acid and urea in a mixed solvent of water and a water miscible organic solvent which has a boiling point of 120° C. or lower, to induce the selective precipitation of S-(−)-amlodipine•D-(−)-tartrate•urea complex of formula (III);

(ii) treating S-(−)-amlodipine•D-(−)-tartrate•urea complex of formula (III) treating with a base in an aqueous solution, to obtain S-(−)-amlodipine of formula (I); and

(iii) optionally, treating S-(−)-amlodipine of formula (I) or S-(−)-amlodipine•D-(−)-tartrate•urea complex of formula (III) with a pharmaceutically acceptable acid in an aqueous solution, to obtain the pharmaceutically acceptable salt of S-(−)-amlodipine of formula (I):

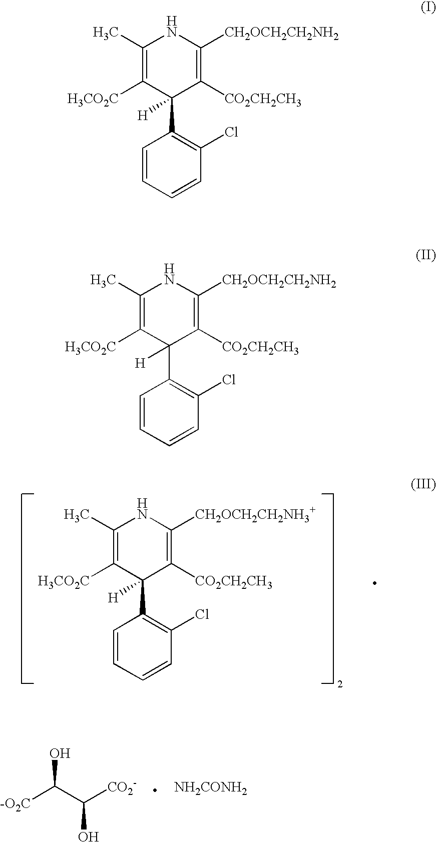

In accordance with another aspect of the present invention, there is provided S-(−)-amlodipine•D-(−)-tartrate•urea complex of formula (III) which can be used as an intermediate in preparing the S-(−)-amlodipine of formula (I).

### DETAILED DESCRIPTION OF THE INVENTION

Hereinafter, the present invention will be described in more detail.

The method for preparing S-(−)-amlodipine according to the present invention is characterized by the selective precipitation of S-(−)-amlodipine•D-(−)-tartrate•urea complex of formula (III) in a mixture of water and a water miscible organic solvent which has a boiling point of 120 r or lower.

In step (i), S-(−)-amlodipine•D-(−)-tartrate•urea complex of formula (III), a key intermediate used in the present invention, is obtained by precipitation. Specifically, S-(−)-amlodipine•D-(−)-tartrate•urea complex of formula (III) is obtained by adding urea and amlodipine in a mixed solvent of a water miscible organic solvent and water, heating and stirring the resulting mixture until it becomes homogenous, to which D-(−)-tartaric acid dissolved in water is added and stirred, followed by cooling the resulting solution to induce the precipitation of S-(−)-amlodipine•D-(−)-tartrate•urea complex of formula (III), and isolating the precipitate by filtrating. The step of heating and stirring is carried out at a temperature ranging from room temperature to 80° C., while the step of cooling to induce precipitation is carried out at a temperature ranging from 5° C. to room temperature preferably with stirring the solution at that temperature for 1 to 24 hours.

The organic solvent used in the present invention may be selected from the group consisting of methanol, ethanol, 1-propanol, 2-propanol, 1-butanol, 2-butanol, t-butanol, methyl acetate, acetonitrile, acetone, methylethylketone, tetrahydrofuran, 1,4-dioxane, and a mixture thereof, which is miscible with water and has a boiling point of 120° C. or lower. The organic solvent may be mixed in an amount ranging from 20 to 80% by volume with water, and the resulting mixed solvent of an organic solvent and water may be used in an amount ranging from 3 to 12 ml based on 1 g of amlodipine. Further, D-(−)-tartaric acid may be used in an amount ranging from 0.25 to 0.5 equivalent based on 1 mole of amlodipine, and urea, in an amount of 0.5 to 5 equivalents based on 1 mole of amlodipine.

The S-(−)-amlodipine component of the crude S-(−)-amlodipine•D-(−)-tartrate•urea complex obtained in step (i) has an optical purity of at least 95% ee.

Also, the crude S-(−)-amlodipine•D-(−)-tartrate•urea complex obtained in step (i) may be optionally subjected to further reprecipitation to obtain a product of higher optical purity by using the mixed solvent.

For example, the reprecipitation is performed by suspending S-(−)-amlodipine•D-(−)-tartrate•urea complex obtained in step (i) in the mixed solvent of the organic solvent and water, heating the suspension at a room temperature to 80° C. until a homogenous solution is obtained, stirring the solution for 30 minutes to 2 hours at 80° C., cooling the resulting solution slowly to a temperature ranging from 5° C. to room temperature, and stirring at that temperature for 1 to 24 hours. The organic solvent used in this reaction may be the same one used in step (i) and it is mixed in an amount ranging from 20 to 80% by volume with water to obtain a mixed solvent, which is preferably used in an amount ranging from 5 to 15 ml based on 1 g of S-(−)-amlodipine•D-(−)-tartrate•urea complex.

The S-(−)-amlodipine component of the reprecipitated S-(−)-amlodipine•D-(−)-tartrate•urea complex has an optical purity of at least 98% ee. The reprecipitation may be performed once again to obtain S-(−)-amlodipine•D-(−)-tartrate•urea complex with an even high optical purity.

S-(−)-amlodipine•D-(−)-tartrate•urea complex of formula (III) obtained in step (i) is a crystalline complex formed by dimolecular S-(−)-amlodipine, monomolecular D-(−)-tartaric acid and monomolecular urea (2:1:1), which has a melting point of approximately 200° C.

In step (ii), an optically pure S-(−)-amlodipine can be obtained by subjecting S-(−)-amlodipine•D-(−)-tartrate•urea complex to neutralization with a base in an aqueous solution.

The preparation of S-(−)-amlodipine of formula (I) comprises treating S-(−)-amlodipine•D-(−)-tartrate•urea complex suspended in water with aqueous sodium or potassium hydroxide to adjust the pH of the solution to 7 to 10. S-(−)-amlodipine released by neutralization can be isolated by extracting with an organic solvent such as dichloromethane or chloroform and concentrating the extract.

Further, S-(−)-amlodipine obtained can be recrystallized in a suitable solvent such as dichloromethane or hexane.

Meanwhile, a pharmaceutically acceptable salt of S-(−)-amlodipine may be one of those disclosed in International Publication Patent Nos. WO 93/10779, WO 03/043989, WO 2004/024689, WO 2006/043148 or WO 2005/058825 and Korea Patent No. 2006/006840 as well as hydrates thereof, among which benzenesulfonate, maleate, nicotinate, camphorsulfonate and hydrates thereof are very important acid addition salts in the art.

The pharmaceutically acceptable salt of S-(−)-amlodipine of the present invention may be prepared in two ways: by reacting S-(−)-amlodipine of formula (I) with an appropriate pharmaceutically acceptable acid based on the prior art as described above; or by reacting S-(−)-amlodipine•D-(−)-tartrate•urea complex of formula (III) with an appropriate pharmaceutically acceptable acid in an aqueous solution.

The pharmaceutically acceptable acid may be selected from the group of consisting of benzenesulfonic acid, maleic acid, nicotinic acid, and camphorsulfonic acid.

For example, S-(−)-amlodipine (1S)-(+)-camphorsulfonate hydrate may be prepared by dissolving S-(−)-amlodipine•D-(−)-tartrate•urea complex in an aqueous solution, e.g., a mixed solution comprising 20 to 60% by volume of water and 40 to 80% by volume of an organic solvent selected from the group consisting of methanol, ethanol, 1-propanol, 2-propanol, acetone, acetonitrile, 1,4-dioxane, and a mixture thereof; adding 1 to 1.1 mole equivalents of (1S)-(+)-camphorsulphonic acid thereto based on 1 mole of S-(−)-amlodipine; adding water until the content of the organic solvent becomes 20% by volume or lower; and filtrating the solid precipitated.

The high optical purity S-(−)-amlodipine or a salt thereof prepared by the method of the present invention can use as an effective therapeutic agent for cardiovascular diseases.

The following Examples are intended to further illustrate the present invention without limiting its scope.

### Example 1

### Preparation of S-(−)-amlodipine•D-(−)-tartrate•urea complex (Compound of Formula (III))

(1-1) 50 g of urea was dissolved in 250 ml of water, added 600 ml of 2-propanol and 112.5 g of amlodipine thereto, and heated to 50° C. Added to the resulting mixture was 10.4 g of D-(−)-tartaric acid dissolved in 50 ml of water, followed by stirring at 50° C. for 1 hour. The resulting solution was slowly cooled to room temperature, and stirred for 15 hours, after which the solution was further cooled to 5° C., and stirred for 3 hours. The precipitate formed was filtered and washed with 2-propanol and dried at 50° C., to obtain a crude S-(−)-amlodipine•D-(−)-tartrate•urea complex as a yellow crystalline powder (51.6 g; yield: 73%).

b.p.: 198.5 to 199.3° C.;

[α]_{D}²⁵: −27.2° (c=0.1, DMF);

optical purity (HPLC): 95.3% ee (enantiomeric excess).

(1-2) 50 g of S-(−)-amlodipine•D-(−)-tartrate•urea complex obtained in (1-1) was suspended in a mixture of 125 ml of 2-propanol and 125 ml of water and heated to 70° C. to obtain a homogenous solution. 250 ml of 2-propanol was added thereto and resulting mixture was slowly cooled to room temperature, and stirred for 18 hours, and then, further cooled to 5° C. and stirred for 3 hours. The precipitate formed was filtered, washed with 2-propanol, and dried at 50° C., to obtain a highly pure form of the title compound as a yellow crystalline powder (45.1 g; yield: 90%).

m.p.: 201.8 to 202.8° C.;

[α]_{D}²⁵: −30.6° (c=0.1, DMF);

S-(−)-amlodipine optical purity (HPLC): 99.8% ee;

tartaric acid content (HPLC): 14.71% (theoretic content: 14.60%);

urea content (HPLC): 5.75% (theoretic content: 5.84%);

¹H-NMR (DMSO-d⁶, ppm): δ 7.35 (d, 2H), 7.22 (m, 4H), 7.10 (m, 2H), 5.42 (br, 4H, urea —NH₂), 5.3 (s, 2H), 4.63 (dd, 4H), 4.0 (q, 4H), 3.88 (s, 2H, tartaric acid-CH(OH)—), 3.61 (t, 4H), 3.50 (s, 6H), 2.97 (t, 4H), 2.31 (s, 6H), 1.10 (t, 6H);

IR (KBr, cm⁻¹): 3499, 3382, 3342, 3217, 2951, 1688, 1635, 1603, 1480, 1423, 1285, 1207, 1104, 1044, 1025.

### Example 2 to 8

### Preparation of S-(−)-amlodipine•D-(−)-tartrate•urea complex (Compound of Formula (III))

The procedure similar to that of Example 1 (1-1) was repeated using the respective organic solvents listed in Table 1, under the condition that the volume of the mixed solvent (an organic solvent:water is 3:1 by volume) was 8 ml per 1 g of amlodipine, while the amounts of D-(−)-tartaric acid and urea were 0.25 and 1 mole equivalent respectively based on 1 mole of amlodipine, to obtain the title compound.

TABLE 1

| | | Optical purity of | |
|---|---|---|---|
| Example No. | Organic solvent | S-(−)-amlodipine (% ee) | Yield (%) |
| 2 | 2-butanol | 98.1 | 64 |
| 3 | t-butanol | 96.2 | 67 |
| 4 | 1-propanol | 98.9 | 25 |
| 5 | 2-propanol | 96.7 | 72 |
| 6 | 1,4-dioxane | 96.3 | 63 |
| 7 | acetonitrile | 94.5 | 68 |
| 8 | methyl acetate | 96.2 | 67 |

### Example 9 to 10

### Preparation of S-(−)-amlodipine•D-(−)-tartrate•urea complex (Compound of Formula (III))

The procedure similar to that of Example 1 (1-2) with the S-(−)-amlodipine•D-(−)-tartrate•urea complex obtained in Example 7 was repeated using the respective organic solvents as listed in Table 2, under the condition that the volume of the mixed solvent (an organic solvent:water is 2:1 by volume) used was 8 ml per 1 g of the complex, to obtain the title compound in purified forms as listed in Table 2.

TABLE 2

| | | Optical purity of | |
|---|---|---|---|
| Example No. | Organic solvent | S-(−)-amlodipine (% ee) | Yield (%) |
| 9 | 2-propanol | 99.8 | 85 |
| 10 | acetonitrile | 99.9 | 82 |

### Example 11

### Preparation of S-(−)-amlodipine (Compound of Formula (I))

20 g of S-(−)-amlodipine•D-(−)-tartrate•urea complex obtained in Example 1 (1-2) was suspended in 200 ml of dichloromethane and 150 ml of water, and adjusted the pH of the resulting solution with 2N sodium hydroxide to 9. The organic layer formed was separated, washed once with 100 ml of water, and dried over magnesium sulfate. The solvent was removed under a reduced pressure, and hexane was added dropwise to the residue, and stirred vigorously to homogenize the precipitate formed. The precipitate was filtered and dried at 40° C. under a reduced pressure, to obtain the title compound as white crystalline powder (14.3 g; yield: 90%).

m.p.: 108 to 110° C.;

[α]_{D}²⁵: −31.9° (c=1.0, MeOH);

S-(−)-amlodipine chiral optical purity (HPLC): 99.9% ee.

### Example 12

### Preparation of (1S)-(+)-camphorsulfonate of S-(−)-amlodipine

10 g of S-(−)-amlodipine obtained in Example 11 was suspended in a mixed solvent of 30 ml of 2-propanol and 30 ml of water, and 5.7 g of (1S)-(+)-camphorsulfonate was added thereto, and the mixture was heated to 40° C. to obtain a homogenous solution. The resulting solution was cooled to room temperature, insoluble materials formed were removed by filtering, and 120 ml of water was added dropwise to the filtrate, and stirred for 4 hours. The precipitate formed was filtered, washed with a mixed solvent of 2-propanol and water (1/5, v/v) and dried at 40° C., to obtain a hydrate form of the title compound as a white crystalline powder (14.5 g; yield: 88%).

m.p.: 146 to 149° C.;

water content: 4.5%;

[α]_{D}²⁵: −7.2° (c=1.0, MeOH);

S-(−)-amlodipine optical purity (chiral HPLC): 99.9% ee;

¹H-NMR (CDCl₃, ppm): δ 7.75 (s, 4H), 7.45-6.09 (m, 4H, ArH), 5.39 (s, 1H), 4.77 (q, 2H), 4.03 (m, 2H), 3.85 (m, 2H), 3.58 (s, 3H), 3.35 (m, 2H), 3.05 (q, 2H), 2.50-2.20 (m, 2H), 2.38 (s, 3H), 2.10-1.80 (m, 3H), 1.75 (m, 1H), 1.38 (m, 1H), 1.15 (t, 3H), 1.00 (s, 3H), 0.80 (s, 3H).

IR (KBr, cm⁻¹): 3431, 3395, 3077, 2953, 1748, 1691, 1643, 1611, 1438, 1289, 1206, 1099, 1041.

### Example 13

### Preparation of (1S)-(+)-camphorsulfonate of S-(−)-amlodipine

20 g of S-(−)-amlodipine•D-(−)-tartrate•urea complex obtained in Example 1 (1-2) was suspended in a mixed solvent of 25 ml of isopropanol and 25 ml of water, 9.07 g of (1S)-(+)-camphorsulfonate was added thereto and heated to 40° C. to obtain a homogenous solution. The insoluble materials were removed by filtering, 200 ml of water was added dropwise to the filtrate and stirred for 4 hours at room temperature. The precipitate formed was filtered, washed with a mixed solvent of 2-propanol and water (1/5, v/v), and dried at 40° C., to obtain a hydrate form of the title compound as a white crystalline powder (23.2 g, yield: 89%).

m.p.: 147 to 150° C.;

water content: 4.5%;

[α]_{D}²⁵: −7.3° (c=1.0, MeOH);

S-(−)-amlodipine optical purity (chiral HPLC): 99.9% ee.

While the invention has been described with respect to the above specific embodiments, it should be recognized that various modifications and changes of the invention also fall within the scope of the present invention defined by the claims that follow.

What is claimed is:

1. A method of preparing S-(−)-amlodipine of formula (I) or a pharmaceutically acceptable salt thereof, which comprises the steps of:
(i) bringing amlodipine of formula (II) to react with D-(−)-tartric acid and urea in a mixed solvent of water and a water miscible organic solvent which has a boiling point of 120° C. or lower, to induce the selective precipitation of S-(−)-amlodipine•D-(−)-tartrate•urea complex of formula (III);
(ii) treating S-(−)-amlodipine•D-(−)-tartrate•urea complex of formula (III) treating with a base in an aqueous solution, to obtain S-(−)-amlodipine of formula (I); and
(iii) optionally, treating S-(−)-amlodipine of formula (I) or S-(−)-amlodipine•D-(−)-tartrate•urea complex of formula (III) with a pharmaceutically acceptable acid in an aqueous solution, to obtain the pharmaceutically acceptable salt of S-(−)-amlodipine of formula (I):

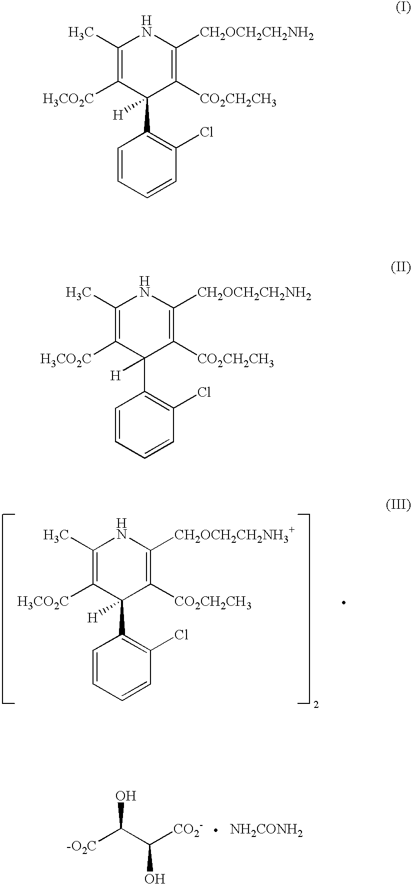

2. The method of claim 1, wherein the organic solvent is selected from the group consisting of methanol, ethanol, 1-propanol, 2-propanol, 1-butanol, 2-butanol, t-butanol, methyl acetate, acetonitrile, acetone, methylethylketone, tetrahydrofuran, 1,4-dioxane, and a mixture thereof.

3. The method of claim 1, wherein the organic solvent content of the mixed solvent is in the range of 20 to 80% by volume.

4. The method of claim 1, wherein the mixed solvent is used in an amount ranging from 3 to 12 ml based on 1 g of amlodipine.

5. The method of claim 1, wherein D-(−)-tartric acid is used in an amount ranging from 0.25 to 0.5 equivalent based on 1 mole of amlodipine.

6. The method of claim 1, wherein urea is used in an amount ranging from 0.5 to 5 mole equivalents based on 1 mole of amlodipine.

7. The method of claim 1, wherein S-(−)-amlodipine•D-(−)-tartrate•urea complex of formula (III) obtained in step (i) is subjected to reprecipitation in the mixed solvent prior to performing step (ii).

8. The method of claim 7, wherein the amount of the mixed solvent used in the reprecipitation is in the range of 5 to 15 ml based on 1 g of S-(−)-amlodipine•D-(−)-tartrate•urea complex of formula (III).

9. The method of claim 1, wherein in step (ii), the base is added to adjust the pH of the reacting solution to a range of 7 to 10.

10. The method of claim 1, wherein the pharmaceutically acceptable salt is selected from the group consisting of benzenesulfonate, maleate, nicotinate, camphorsulfonate and hydrates thereof.

11. The method of claim 10, wherein the pharmaceutically acceptable salt is (1S)-(+)-camphorsulfonate.

12. The S-(−)-amlodipine•D-(−)-tartrate•urea complex of formula (III): [chemical structure]
